(19) United States
(12) Patent Application Publication
(10) Pub. No.: US 2026/0351537 A1
(43) Pub. Date: Nov. 5, 2026

(54) READY-TO-USE CALIBRATION COMPOSITION AND METHOD FOR CALIBRATING A MASS SPECTROMETER

(71) Applicant: BIOMÉRIEUX, Marcy L'Etole (FR)

(72) Inventors: Jean-Philippe CHARRIER, Tassin La Demi Lune (FR); Rémi-Vinh COUDERT, Lyon (KR); Frédéric JAUFFRIT, Lyon (KR); Nadine PERROT, Montluel (FR)

(73) Assignee: BIOMÉRIEUX, Marcy L'Etole (FR)

(21) Appl. No.: 19/486,198

(22) PCT Filed: Jun. 7, 2024
(86) PCT No.: PCT/FR2024/000063

§ 371 Date: Nov. 20, 2025

(30) Foreign Application Priority Data
Jun. 9, 2023 (FR) FR2305868

(51) Int. Cl. H01J 49/00; H01J 49/04; H01J 49/16

(52) U.S. Cl. CPC H01J 49/0009; H01J 49/0418; H01J 49/164

(57) ABSTRACT

A ready-to-use calibration composition for a mass spectrometer, including whole E. coli bacteria suspended in a liquid which is based on alcohol(s) and/or acetonitrile and is sanitizing, volatile and does not denature bacterial proteins.

The present invention relates to the field of mass spectrometry, which finds various applications in the fields of biomedical research, medicine, diagnostics and biotechnology. More precisely, the invention relates to a calibration composition and to a method for calibrating a mass spectrometer using the matrix-assisted desorption/ionization technique, known as MALDI. In particular, the invention is suitable for the matrix-assisted desorption/ionization time-of-flight technique, known as MALDI-TOF. The aim of the calibration composition and the calibration method according to the invention is to make it possible to ensure a reliable calibration of the spectrometer used and thus to obtain sufficiently precise measurements of the masses of the ions generated from a sample to be analyzed.

The invention also relates to the use of a calibration composition according to the invention as an external standard for calibrating a mass spectrometer and to a method for characterizing a sample containing at least one microorganism.

The analysis of microorganisms and, more generally, the analysis of a sample liable to contain a microorganism by mass spectrometry and, in particular, by MALDI or MALDI-TOF has been used for a number of years to carry out rapid identification of microorganisms, and also for determining the resistance of microorganisms to certain antimicrobial agents, as illustrated, for example, in patent application WO 2016/0166580, in the name of the applicant. The identification of a microorganism is carried out on the basis of the mass spectrum of the most abundant proteins in the microorganism, by comparison with reference data allowing, in particular, the identification of the family, the genus and usually the species of the microorganism.

The protocol used routinely comprises depositing a sample to be analyzed, which is liable to contain a microorganism, on a MALDI analysis plate, adding a matrix suitable for the MALDI technique, acquiring the mass spectrum using a MALDI-TOF mass spectrometer, and identifying the species by comparison with reference data stored in a database. The MALDI-TOF technique is also used for detecting the resistance of a microorganism to an antibiotic, and in particular for identifying a phenotype responsible for the hydrolysis of beta-lactam antibiotics due to the secretion of beta-lactamase enzymes, and in particular carbapenemase enzymes.

Various mass spectrometers suitable for such a characterization are sold in particular by the applicant (in particular under the references VITEK® MS and VITEK® MS Prime), and also by the companies Bruker Daltonics and Autobio in particular. These spectrometers are of the MALDI-TOF type and comprise in particular a laser ionization source and a time-of-flight mass analyzer. They are intended to operate with an analysis plate, also called a MALDI plate or MALDI target, on which a sample to be analyzed is deposited in combination with a matrix suitable for the MALDI technique, it being possible for the latter to be deposited at the same time as or after the sample.

Then, the analysis plate is introduced into an analysis chamber of the spectrometer, this chamber being brought to a relatively high level of vacuum, with a pressure which is for example less than 10⁻⁵ mbar (10⁻³ Pa), typically in the range extending from 10⁻⁶ to 10⁻⁹ mbar (10⁻⁴ to 10⁻⁷ Pa). Under these vacuum conditions, the sample placed within the MALDI matrix is subjected to soft laser ionization. The matrix then absorbs photon energy and the restoration of this energy leads to the sublimation of the matrix, the desorption of the molecules present in the sample and the appearance of matter in a state called a plasma. Within this plasma, charge exchanges occur between molecules from the matrix and molecules from the sample, and in particular microorganisms. For example, protons can be stripped from the matrix and transferred to the proteins, peptides and organic compounds present in the sample.

This step enables gentle ionization of the molecules present without causing their destruction. The sample thus releases ions of various sizes. In MALDI-TOF spectrometers, the ions generated are then accelerated by an electric field and fly freely in a tube under reduced pressure, called a flight tube. The smallest ions will then “travel” faster than the larger ions, allowing them to be separated. A detector is located at the terminal end of the flight tube. Thus, a mass spectrum is obtained, representing the intensity of the signal corresponding to the number of ionized molecules of a given mass-to-charge [m/z], as a function of the time of flight (TOF) of the molecules striking the detector. The flight time taken by the ions is used to calculate their mass-to-charge ratio [m/z], expressed in thomsons (Th).

The calibration of a mass spectrometer makes it possible to increase the reliability of the measurements performed and therefore the utilization of the mass spectra generated, especially for the identification of microorganisms in a sample. The calibration consists in adjusting the experimental mass-to-charge value obtained to a “calibrated” value, which will then appear in the generated mass spectrum and be used for characterizing the sample to be analyzed.

Such calibrations can be carried out by using an internal calibrating agent integrated into the sample, or an external calibrating agent, called an external standard, deposited on at least one deposition zone of a MALDI analysis plate.

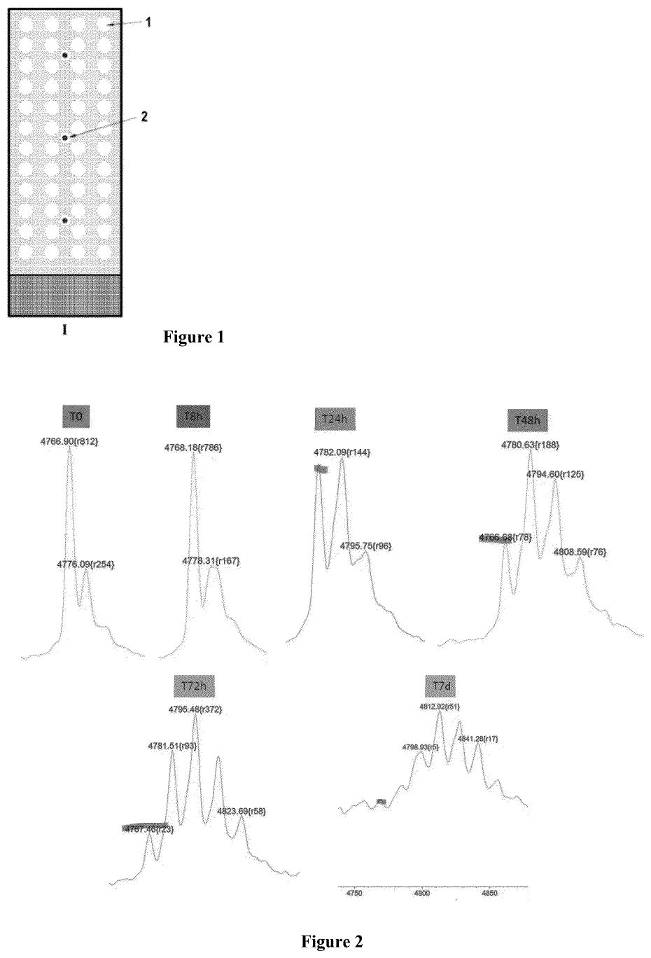

As illustrated in FIG. 1, a MALDI analysis plate I typically comprises:
a series of deposition zones 1 (also called spots), referred to as analysis zones, for the deposition of sample(s) to be characterized; after drying, these zones 1 form what are referred to as characterization zones, and
one or more deposition zones 2, referred to as reference zones, for the deposition of an external standard; after drying, these zones 2 form what are referred to as control zones;
   these zones are in the form of wells, usually circular in shape.

MALDI plates are generally manufactured from materials such as stainless steel, gold, silicon, a plastic polymer coated with metal, or enriched with a conductive agent (such as carbon black), or coated glass. They generally have a textured surface to facilitate the deposition of the samples to be analyzed and the external standards.

In order to promote subsequent ionization, the surface of the plate is generally conductive, at least at the analysis and reference deposition zones. By way of example, such an analysis plate is formed of a polymer such as polypropylene, said polymer being covered with a layer of stainless steel. The polymer may contain a conductive material such as carbon black. Various MALDI plates are commercially available, such as VITEK® MS plates from bioMérieux (disposable) and MALDI Biotarget plates from Bruker Daltonics (reusable). Such plates I usually comprise from 48 to 96 analysis zones 1, and at least one, or even two or three, reference zones 2, the size of which may be different from that of the analysis zones 1.

Currently, for the calibration of its apparatuses, the applicant recommends using a freshly cultured Escherichia coli (E. coli) ATCC 8739 strain as external standard, as described in the VITEK® MS user manual. The bacteria are cultured on agar medium at 37 °C for 18 to 24 hours. An amount of bacterium is then recovered and then deposited on the MALDI plate, at a reference zone. This calibration method has the inconvenience of having to have available freshly cultured bacteria.

In addition, taking bacteria from an agar culture in order to deposit them on a MALDI analysis plate, in a specifically dedicated zone of a few mm² (in particular 5 to 15 mm²), and forming a thin and homogeneous layer of cells, proves to be a particularly delicate manual operation. The quality of the results can vary greatly from one operator to another, from one operation to another. However, if the external standard deposit is of poor quality, the identification of a microorganism on a MALDI analysis plate that comprises a large number of spots for the deposition of a sample of interest (usually 16 to 96 analysis spots) may fail. The operator is then forced to completely redo the plate preparation.

Other types of external standard that make it possible to carry out a calibration of a mass spectrometer are proposed in the prior art. By way of example, BTS (for Bacterial Test Standard), sold by Bruker, is an extract of E. coli enriched with two proteins of high molecular weight. BTS was developed to serve as quality control for Bruker's MALDI-TOF Biotyper spectrometer. The BTS data sheet indicates that its specific composition covers the entire protein mass range used by the Biotyper for the precise identification of microorganisms, and in particular the range extending from 3.6 to 17 kDa. With the BTS, the Biotyper performs automatic quality control before each identification cycle. The quality control process comprises calibration of the mass spectrometer, verification of the tuning of the laser, and evaluation of the quality of the spectrum. The performance of the system is finally confirmed by the identification of E. coli, which must meet minimum performance criteria. Before being used, a dose of BTS is placed in approximately 50 μL of a solvent composed of 50% v/v (volume to the total volume of solvents) acetonitrile, 47.5% v/v water and 2.5% v/v trifluoroacetic acid. The mixture is incubated for 5 minutes at ambient temperature and various operations of mixing by pipetting and centrifugation are recommended by the supplier Bruker in document Ref. 8290190, September 2017, “IVD Bacterial Test Standard”. 5 μL aliquots thus obtained can be stored in stoppered tubes, frozen at −18 °C or less, and then used to form a deposit on four different deposition zones of a MALDI plate.

Mention may also be made of patent CN104931572B. The latter proposes a calibration composition which, like Bruker's BTS, is of the bacterial extract type, but with the advantage for its user of being ready to use. This bacterial extract/lysate is prepared according to the following method, according to the available machine translation:
(1) select E. coli strains ATCC 8739, MG 1655, JM 109, culture them up to their logarithmic growth phase, mix them according to the mass ratio 3-8:1-5:1-3;
(2) a protein washing solution A, which is deionized water or ultrapure water, is added to the mixture of strains in a 5-10 mg: 0.1-0.5 mL mass/volume ratio, and then a protein washing solution B, which is anhydrous ethanol, is added in a 5-2:1 volume ratio; after mixing and centrifugation, the supernatant is removed;
(3) add, to the precipitate obtained in step (2), a protein dissolving solution A in a (5-10 mg):(0.025-0.125 mL) mass/volume ratio, mix well, and then add the same volume of a protein dissolving solution B as for the protein dissolving solution A; centrifuge and take the supernatant as the calibration composition; the protein dissolving solution A is a 50% to 80% formic acid solution of HPLC grade and the protein dissolving solution B is HPLC grade acetonitrile.

It is therefore clear that this calibration composition is complex to manufacture; it involves multiple delicate preparation operations.

In view of the calibration compositions of the prior art and the noted deficiencies, the present invention proposes to provide novel calibration compositions that combine the following advantages:
ease of use and handling, in particular in terms of the operation of collecting and depositing an appropriate amount of bacteria in a thin and homogeneous layer, which does not require any particular experience or skill from the user,
ready-to-use formulations having a sufficiently long shelf life,
simple and inexpensive industrial production.

In this context, the present invention proposes a calibration composition for a mass spectrometer, comprising whole E. coli bacteria suspended in a liquid which is based on alcohol(s) and/or on acetonitrile.

For the proposed application, the inventors have thus observed and demonstrated that alcohols of the “unsaturated aliphatic alcohol” type (such as methanol, ethanol, propanol and butanol) and acetonitrile were particularly advantageous because of their good volatility, their asepticizing properties that are non-denaturing for the bacteria, and their chemical/reactive neutrality with regard to the proteins. Consequently, a calibration composition according to the invention is formulated in the form of an intrinsically volatile, asepticizing liquid, which enables good and lasting preservation of the bacteria and their proteins.

For the purposes of simplifying the text, in the present description, the term “alcohol” denotes an unsaturated aliphatic alcohol, in particular an unsaturated C1-C4 aliphatic alcohol, preferably ethanol and isopropanol.

A calibration composition according to the invention thus contains whole bacteria, preserved in this state until the moment of initiating the calibration operations. Maintained in such an environment, the bacterial proteins, in particular those chosen as references for calibration, enjoy protection against biological and/or chemical degradations, provided both by the structure itself of the bacteria, which remain whole/intact, and by the alcohol(s) and/or acetonitrile. While exerting an asepticizing effect on the entire composition, the alcohol(s) and/or acetonitrile of a calibration composition according to the invention exhibit, as such, an advantageous chemical/reactive neutrality with regard to the proteins.

In addition to ensuring good and lasting preservation of the bacteria and their proteins, such a liquid and volatile formulation imparts the calibration compositions according to the invention with the advantage of being able to be used directly and as they are, and of greatly facilitating the spectrometer calibration operations. With a calibration composition according to the invention, it will suffice for the operator to take the prescribed volume (which corresponds to a pre-established amount of cells), and to deposit it on the analysis plate. To do so, it will be possible to use a pipette or any other suitable utensil. The high volatility of the alcohol(s) and/or acetonitrile which make(s) up the liquid phase of a calibration composition according to the invention allow(s) the liquid phase to evaporate rapidly, leaving only a thin and homogeneous layer of cells on the deposition zone. Once the matrix has been added and the analysis plate loaded into the spectrometer, data acquisition can be started. Similarly, it is possible to mix the calibration composition with the matrix, then deposit a volume of this mixture on the analysis plate, and load it into the spectrometer once the mixture is sufficiently dry.

Advantageously, the liquid phase of a calibration composition according to the invention is based on alcohol(s). According to a preferred embodiment, it is an aqueous liquid with a content of alcohol(s) of at least 20% v/v, typically 70% v/v.

Advantageously and according to the invention, the alcohol(s) included in the calibration composition according to the invention is/are ethanol and/or isopropanol. According to a particularly preferred embodiment, the liquid based on alcohol(s) of a calibration composition according to the invention consists of an aqueous water/ethanol mixture, with an ethanol content of 70%.

According to another preferred embodiment, the liquid phase of a calibration composition according to the invention is based on acetonitrile, that is to say a 100% v/v acetonitrile solution or else a water/acetonitrile mixture, with a content of acetonitrile of at least 20% v/v, typically of 70% v/v.

Advantageously, in order to have a composition that is as simple as possible, the E. coli bacteria of a calibration composition according to the invention are obtained from a single strain. In particular, these bacteria are obtained from the BL21 strain or from the ATCC 8739 strain. The BL21 E. coli strain was described in 1986 (Studier F. W., Moffatt B A., J. Mol Biol. 1986 May 5; 189 (1): 113-30. “Use of bacteriophage T7 RNA polymerase to direct selective high-level expression of cloned genes”), and was characterized in its entirety by sequencing in 2015 (Jeong H., Ju Kim H., Jun Leeb S., Genome Announc. 2015 Mar. 19; 3 (2). e00134-15. Complete Genome Sequence of Escherichia coli Strain BL21). This strain of E. coli is commercially available and can be obtained from Agilent (catalog reference #200133). The ATCC 8739 strain is also commercially available, notably from the ATCC. Its genetic sequence is available in GenBank under number GCA_016864475.1. Although not preferred, instead of the strain BL21 or ATCC 8739, it is also possible to use any other E. coli strain known to a person skilled in the art, in particular the K-12 E. coli strain (ATCC 10798).

Advantageously and according to the invention, the following calibration compositions are particularly preferred:
a calibration composition consisting of a suspension of E. coli bacteria of the BL21 strain in an aqueous liquid based on alcohol and/or on acetonitrile.
a calibration composition consisting of a suspension of E. coli bacteria of the ATCC 8739 strain in an aqueous liquid based on alcohol and/or on acetonitrile.

In the calibration compositions according to the invention, the composition of alcohol(s) and/or acetonitrile is chosen for structural preservation of the E. coli bacteria present in the composition, at a temperature of 0 to 37 °C, in particular from 0 to 25 °C.

In particular, advantageously, this aqueous liquid based on alcohol(s) and/or on acetonitrile, or more generally the calibration composition, does not comprise any acid or other chemical substance capable of reacting with the proteins, directly or indirectly. Preferably, any chemical substances (such as detergents, bases) that are capable of extracting proteins that are not usually detected with a conventional deposit will be avoided.

In particular, in the calibration compositions according to the invention, the E. coli bacteria are present at a concentration of 2 to 7 McF (McFarland), in particular 3 to 6 McF, for example 5 McF. Such a concentration can be determined by a densitometer (for example, the DENSIMAT sold by bioMérieux), which makes it possible to measure the optical density of a liquid composition, in particular under incident light with a wavelength between 500 and 600 nm, and preferably of 550 nm.

The concentration of bacteria in a liquid composition can also be expressed in colony-forming units per milliliter (cfu/mL). Thus, advantageously, in the calibration compositions according to the invention, the E. coli bacteria are present at a concentration of from 6×10⁸ to 21×10⁸ cfu/mL.

Table 1 below gives the correspondence between the optical density, the value in McFarland units (McF) and the bacterial concentration in 10⁸ cfu/mL.

TABLE 1
Correspondence between McFarland units and cfu/mL

| | | Theoretical |
|---|---|---|
| McFarland | Bacterial | optical |
| scale | concentration | density |
| standard | 10⁸ cfu/mL | at 550 nm |
| 0.5 | 1.5 | 0.125 |
| 1 | 3 | 0.25 |
| 2 | 6 | 0.50 |
| 3 | 9 | 0.75 |
| 4 | 12 | 1.00 |
| 5 | 15 | 1.25 |
| 6 | 18 | 1.50 |
| 7 | 21 | 1.75 |

The calibration compositions according to the invention are stable. This stability can be demonstrated by mass spectrometry, in particular by MALDI-TOF. Thus, no degradation of the proteins present, in particular the proteins chosen as references for the calibration, is observed. Their masses, to within analytical error, do not change. The stability of the calibration compositions according to the invention can also be verified through their capacity to provide calibrations of relatively constant, high quality over time.

Because of their good stability, the calibration compositions according to the invention can be stored in stoppered tubes, especially plastic tubes, or in any other type of suitable and hermetically closed container. They can then be stored in this way for several months, in particular for at least 1 month, or at least 2 months, and preferably for at least 6 months, at a temperature of 0 to 37 °C, typically from 0 to 25 °C. Advantageously, it is not necessary to store the compositions according to the invention in frozen form, at negative temperatures, in particular at −18 °C.

Thus, without taking special storage precautions, the calibration compositions according to the invention result in deposits on MALDI plates which are reliable and reproducible and therefore make it possible to carry out calibrations, and therefore mass spectrometry measurements, on samples or microorganisms to be characterized, which are just as reliable and reproducible.

The calibration compositions according to the invention are ready-to-use solutions that can easily be used during mass spectrometry measurements and/or calibration procedures. Because of their liquid formulation, they are easy to collect and deposit. Evaporation of the liquid phase then gives way to a localized deposit, in the form of a thin and uniform cell layer. In particular, a precise volume of 1 to 2 μL, and typically 1 μL, of a calibration composition according to the invention can be easily and rapidly collected and then deposited on the analysis plates, precisely on the deposition zones. The calibration compositions of the invention simplify the calibration workflow to be performed during mass spectrometry measurements, by avoiding the need to perform new E. coli cultures each day, since the calibration compositions are easily stored in a ready-to-use form.

According to another of its subjects, the invention relates to the use of a calibration composition according to the invention as an external standard for calibrating a mass spectrometer according to the MALDI technique, and in particular according to the MALDI-TOF technique.

The calibration compositions according to the invention could also be used in internal calibration, for example to adjust the acquisition parameters of the mass spectrometers according to the MALDI technique, and in particular according to the MALDI-TOF technique used, or as quality control.

Within the context of the invention, also provided is a method for calibrating a mass spectrometer suitable for the MALDI technique, and in particular for the MALDI-TOF technique, comprising the following steps:
a) providing a calibration composition according to the invention,
b) depositing said calibration composition and a matrix suitable for the MALDI technique on at least one deposition zone of a MALDI analysis plate, in order to obtain a control zone after drying,
c) placing said MALDI analysis plate in the mass spectrometer and obtaining a mass spectrum for the control zone according to the MALDI technique, and in particular according to the MALDI-TOF technique, over at least one determined masses-to-charge range and using the experimental values obtained for the masses-to-charge of peaks of said spectrum corresponding to ions of so-called reference proteins of the calibration composition, as calibration values,
d) calculating a calibration relationship based on the theoretical masses-to-charge of the ions of the reference proteins and the calibration values.

Advantageously, the determined masses-to-charge range covers at least the range from 2000 Th to 20 000 Th, preferably from 2000 Th to 15 000 Th.

As a specific embodiment of the method according to the invention, the theoretical masses-to-charge used in step d) may be at least 6 in number, preferably from 8 to 50 in number, and most particularly from 8 to 32. According to one particular embodiment, when the strain of the calibration composition is an ATCC 8729 E. coli strain, the ions of the reference proteins and therefore the theoretical masses-to-charge in Th used are chosen from those listed in Table 2.

According to another particular embodiment, when the strain of the calibration composition is a BL21 E. coli strain, the ions of the reference proteins and therefore the theoretical masses-to-charge in Th used are chosen from those listed in Table 5.

According to specific embodiments, the theoretical masses-to-charge used in step d) are at least 6 in number, preferably 8 to 50 in number, and are, in particular, chosen from the following theoretical masses-to-charge m/z in Th:
when the E. coli strain is BL21: 3128.18; 3158.57; 3179.59; 3206.28; 3579.84; 3637.70; 3936.51; 4163.59; 4185.35; 4365.31; 4438.61; 4449.60; 4497.60; 4613.75; 4768.45; 4777.56; 5069.74; 5096.78; 5150.50; 5381.35; 5461.10; 5612.32; 5725.60; 6255.36; 6298.04; 6316.14; 6411.55; 6508.45; 6547.83; 6857.81; 7158.68; 7274.39; 7707.56; 7872.02; 8326.18; 8369.69; 8876.22; 8898.19; 8994.20; 9060.27; 9226.49; 9535.89; 9554.12; 9741.83; 10 138.48; 10 300.00; 11 223.63; 11 450.19 and 12 227.19;
when the E. coli strain is ATCC 8739:3128.18; 3158.57; 3206.28; 3579.84; 3637.70; 4163.59; 4185.35; 4365.31; 4438.61; 4613.75; 4768.45; 4777.56; 5069.74; 5096.78; 5150.50; 5381.35; 5612.32; 6255.36; 6316.14; 6411.55; 7158.68; 7274.39; 8369.69; 8876.22; 8994.20; 9226.49; 9535.89; 9554.12; 10 138.48; 10 300.00; 11 223.63 and 11 450.19.

More specifically still, the theoretical masses-to-charge used in step d) are at least 6 in number, preferably 8 to 32 in number, and particularly 32 in number, and are, in particular, chosen from the following theoretical masses-to-charge m/z in Th, when the E. coli strain is BL21: 3128.18; 3158.57; 3206.28; 3579.84; 3637.70; 4163.59; 4185.35; 4365.31; 4438.61; 4613.75; 4768.45; 4777.56; 5069.74; 5096.78; 5150.50; 5381.35; 5612.32; 6255.36; 6316.14; 6411.55; 7158.68; 7274.39; 8369.69; 8876.22; 8994.20; 9226.49; 9535.89; 9554.12; 10 138.48; 10 300.00; 11 223.63 and 11 450.19.

The MALDI matrix may be any matrix suitable for the MALDI technique. In particular, such a matrix comprises one of the following compounds: 3,5-dimethoxy-4-hydroxycinnamic acid, α-cyano-4-hydroxycinnamic acid, ferulic acid and 2,5-dihydroxybenzoic acid.

Lastly, according to another of its aspects, the invention relates to a method for characterizing a sample containing at least one microorganism on the basis of the acquisition of a mass spectrum, by the MALDI technique, and in particular by the MALDI-TOF technique, implementing the following steps:
i) depositing said sample and a matrix suitable for the MALDI technique on at least one deposition zone of an analysis plate, and drying, in order to obtain a characterization zone,
ii) placing said analysis plate in a mass spectrometer suitable for the MALDI technique, and in particular for the MALDI-TOF technique, and generating a mass spectrum for the characterization zone,
iii) applying the calibration relationship calculated according to the calibration method of the invention for said mass spectrometer, and determining the mass-to-charge of the peaks in the generated mass spectrum.

In particular, the control analysis zone and the characterization zone are part of the same MALDI analysis plate. This has the advantages of making the calibration operations simpler and faster by reducing the handling steps.

According to a specific embodiment, the characterization method according to the invention comprises identifying a microorganism present in the sample, by comparing the mass-to-charge values of at least some peaks of the generated mass spectrum obtained in step iii) with reference values characteristic of the microorganism.

Other objectives, features and advantages of the invention will emerge in the light of the description that follows and the examples developed below, which refer to the appended figures in which:

FIG. 1 represents a schematic top view of a MALDI plate, in particular sold by the applicant;

FIG. 2, in conjunction with example 2, shows the evolution over time of the mass spectrum by MALDI-TOF obtained with a VITEK® MS system, for an E. coli composition outside of the invention, the bacteria being suspended whole in a commercial mixture of solvents (in this case, the HCCA matrix from bioMérieux; ref. 411071);

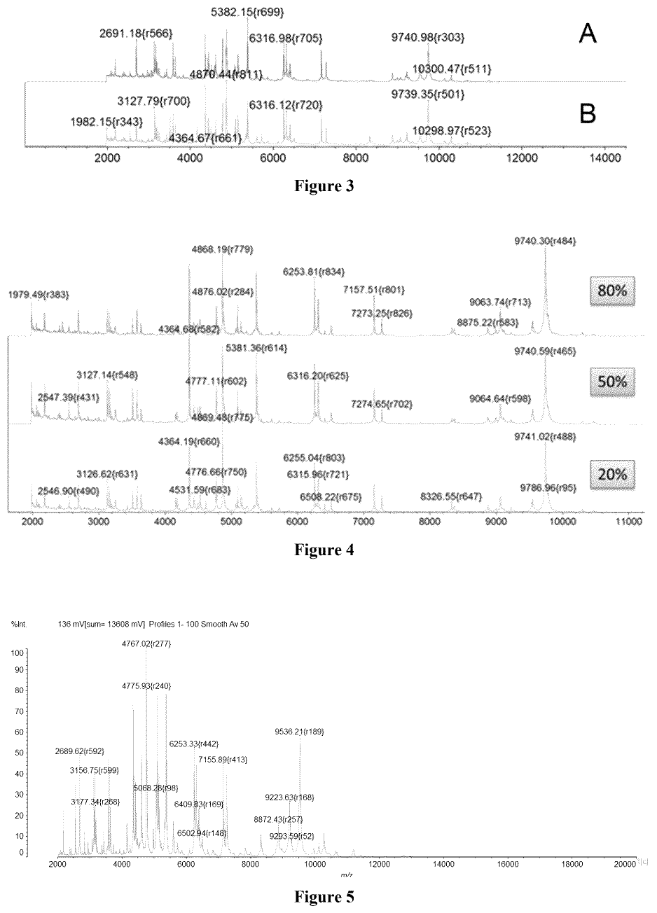

FIG. 3, in conjunction with example 3, shows the spectra obtained with a VITEK® MS system, for two calibration compositions according to the invention, containing 70% v/v ethanol, after a storage period of five months at ambient temperature; these compositions having been prepared according to 2 different protocols (A: from E. coli ATCC 8739 cultured on agar medium; B: from E. coli ATCC 8739 cultured in culture broth);

FIG. 4, in conjunction with example 4, shows different MALDI-TOF spectra obtained with a VITEK® MS system, corresponding to the analysis of the contents of a tube initially comprising 100 μL of a calibration composition according to the invention (E. coli ATCC 8739 suspended in an aqueous liquid containing 70% v/v ethanol), after evaporation of 20%, 50% or 80% of the initial volume;

FIG. 5, in conjunction with example 6, shows a MALDI-TOF mass spectrum obtained with a VITEK® MS system, before calibration, for a calibration composition of example 3 (E. coli BL21 suspended in an aqueous liquid containing 70% v/v ethanol);

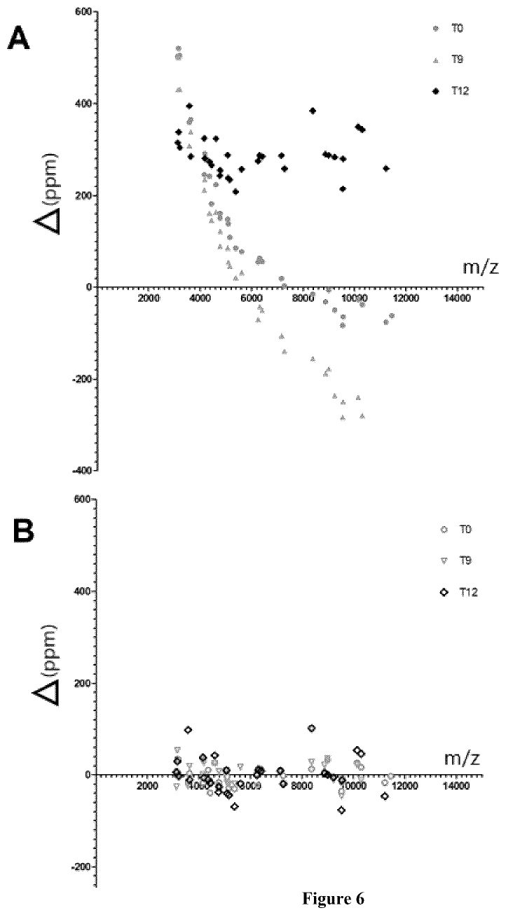

FIG. 6, in conjunction with example 7, is two graphical representations of the errors attributed to the peaks (m/z) chosen as references for the calibration, obtained for a calibration composition according to the invention (E. coli BL21 suspended in an aqueous liquid containing 70% v/v ethanol), freshly prepared (TO), or after 9 or 12 months of storage at 2-8 °C (T9, T12); the associated MS spectra having been acquired with a VITEK® MS system before calibration (A) and after calibration (B);

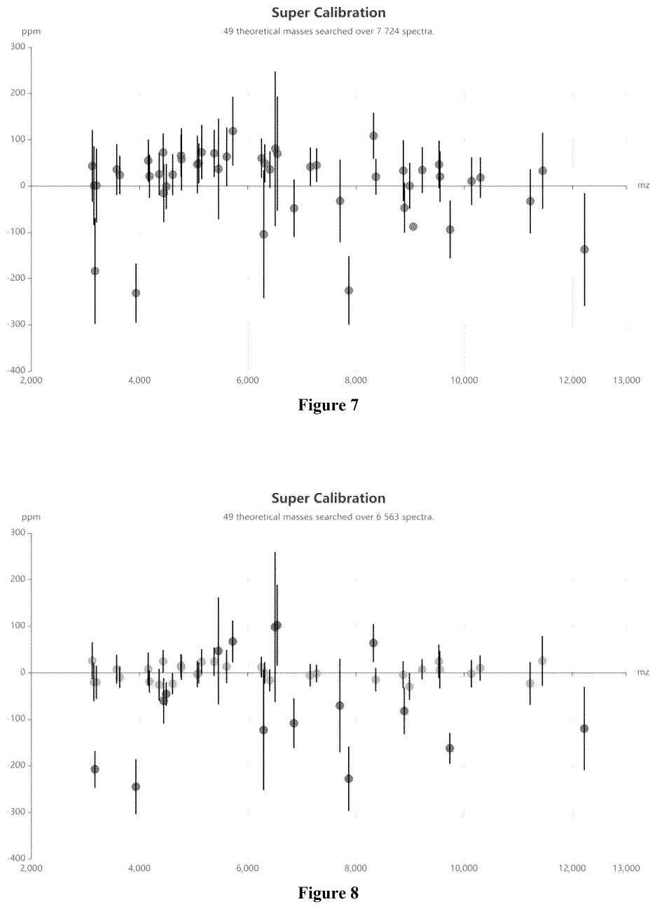

FIG. 7 shows the first super calibration graph used in example 8;

FIG. 8 shows the second, final super calibration graph from the 49 theoretical masses selected over the 6563 MALDI-TOF spectra obtained with a VITEK® MS system, used in example 8.

### EXAMPLE 1: PREPARATION OF A CALIBRATION COMPOSITION ACCORDING TO THE INVENTION, DEPOSITION ON A MALDI ANALYSIS PLATE, AND CALIBRATION

A calibration composition according to the invention can be prepared by simply placing a chosen amount of whole E. coli bacteria of the selected strain in a liquid based on alcohol(s) and/or on acetonitrile. The calibration composition thus comprises a suspension of preserved whole E. coli bacteria. Depending on the amount of calibration composition to be prepared, the E. coli bacteria used may be those directly purchased or derived from bacteria purchased and cultured, according to any suitable technique: on Petri dish, in broth, etc. These culture techniques are well known to a person skilled in the art. As demonstrated in the examples, the culture method has no influence on the stability of the calibration composition obtained.

As an example of a method for preparing a calibration composition according to the invention, E. coli bacteria are cultured in broth, in a fermenter, until the desired amount of biomass has been achieved. The biomass is then pelleted by centrifugation and then diluted in 70% ethanol solution in order to obtain the desired concentration. In order to facilitate the homogenization of the solution and correctly quantify the bacterial concentration, a mechanical dispersion operation may prove to be useful. This can be achieved, for example, by creating a vortex or by dispersing the bacteria with an instrument such as an Ultra-Turrax from IKA (Staufen, Germany) equipped with a high-performance rotor-stator. A sonication step could also be performed. In all cases, care must be taken to minimize the degradation of bacterial cells to avoid rupturing membrane walls and forming cell debris. Despite the precautions taken, some bacteria may be degraded and cell debris may be found in the calibration compositions according to the invention. Once the solution has been homogenized and adjusted to the desired concentration, for example 5 McF, all that remains is for it to be distributed in a suitable container to facilitate its use.

A calibration composition according to the invention may be used directly or may be stored in any suitable container and/or be sold as a ready-to-use composition in any suitable container. As suitable container, mention may in particular be made of a stoppered tube, for example made of plastic, or any type of suitable hermetically closed container. The container will have a volume selected so as to be able to contain the desired amount of calibration composition, in particular from 10 to 300 μL and typically 200 μL. Such volumes make it possible to produce a large number of deposits, which are typically 1 μL deposits, and therefore a large number of calibrations.

The preliminary stability studies presented in the examples have shown that the calibration compositions according to the invention are stable, in particular for at least one year at 2-8 °C, at least two months at ambient temperature (typically at 18-25 °C) when they are stored in a closed container, and for at least one day at ambient temperature when the container is open. The calibration compositions according to the invention are therefore stable over time and make it possible to obtain a good calibration.

During its use, a calibration composition according to the invention is deposited at the control zone(s) of a MALDI plate. This deposition can be carried out using a pipetting technique, or possibly a spraying technique. A drop, for example of about 1 to 2 μL, of the calibration composition may be deposited so as to cover the entire deposition zone. It is easy to form a thin and uniform layer of calibration composition on the plate due to the liquid formulation.

The use of a calibration composition according to the invention does not require any particular expertise or skill. They are compatible with manual and automated preparation (for example using the Colibri® technique offered by the applicant) of MALDI plates, and in particular of the VITEK® MS-DS type.

The operation consisting in depositing a calibration composition according to the invention and a matrix suitable for the MALDI technique on the MALDI plate can be done sequentially or by depositing a mixture of said calibration composition and the matrix.

Advantageously, it will be performed as follows: deposition of the calibration composition, deposition of the MALDI matrix, then drying. The calibration composition according to the invention can be simply deposited by pipetting 1 μL onto a control zone of the MALDI plate, followed by the addition of 1 μL of MALDI matrix, in particular HCCA matrix, and drying. The matrices used in the MALDI technique are generally organic molecules having specific characteristics. They must be able to absorb the energy of the laser used for the irradiation, resulting in the vaporization of the matrix and the formation of ions. In addition, the matrices must be compatible with the samples analyzed and must not interfere with the ions of interest. Generally, the matrices used in the MALDI technique are photosensitive and crystallize in the presence of the population of microorganisms, while preserving the integrity of the molecules present. Such matrices, in particular suitable for the MALDI-TOF mass spectrometry technique, are well known. They are constituted, for example, of a compound chosen from: 3,5-dimethoxy-4-hydroxycinnamic acid, α-cyano-4-hydroxycinnamic acid (present in the HCCA matrix), ferulic acid and 2,5-dihydroxybenzoic acid. Many other compounds are known to a person skilled in the art. There are also liquid matrices which neither crystallize at atmospheric pressure nor even under reduced pressure. Any other compound making it possible to ionize the molecules present in the characterization zone under the effect of a laser beam can be used. Each matrix has specific properties that make it suitable for different types of samples and analyses.

For the constitution of the matrix, such a compound is dissolved, usually in water, preferably of “ultrapure” grade, or in a water/organic solvent(s) mixture. As examples of organic solvents conventionally used, mention may be made of acetone, acetonitrile, methanol or ethanol. The matrix may contain trifluoroacetic acid, which facilitates ionization. The matrix is deposited directly on the control zone and covers or is mixed with the calibration composition.

Drying of the deposited calibration composition, before or after deposition of the matrix, on the MALDI plate is carried out at a temperature and for a time that are suitable for ensuring the formation of a thin and uniform layer of the latter. Preference is given to mild conditions well known to a person skilled in the art. The drying of the calibration composition, alone and/or with a matrix added, can be carried out by evaporation by exposing the whole to ambient air for a few minutes. This evaporation also ensures the crystallization of the matrix present.

Advantageously, the amount of E. coli bacteria of the calibration composition deposited per control zone is generally at least 10⁵ cfu, and in particular from 6×10⁵ to 42×10⁵ cfu.

Once the MALDI plate has been prepared with a layer of calibration composition, in the form of a control zone as described above, it is placed in a mass spectrometer, in particular MALDI or MALDI-TOF mass spectrometer, and a corresponding mass spectrum is acquired. This involves irradiating the MALDI plate with a MALDI laser, which results in desorption and ionization of the molecules present in the irradiated spot. The desorbed and ionized proteins are then detected in order to generate the mass spectrum. The acquisition of the mass spectrum is therefore carried out by irradiating the MALDI plate with a MALDI laser and detecting the desorbed and ionized proteins.

In particular, any type of MALDI-TOF mass spectrometer may be used for producing the mass spectrum. Such apparatuses comprise:
i) an ionization source (generally a UV laser) intended to ionize the control zone and thus the calibration composition and/or a sample present on a characterization zone;
ii) an accelerator of ionized molecules by application of a potential difference;
iii) a tube under reduced pressure, in which the ionized and accelerated molecules travel;
iv) a mass analyzer intended to separate the molecular ions formed, as a function of their mass-to-charge (m/z);
v) a detector intended to measure the signal produced directly by the molecular ions.

The laser beam used for the ionization may have any type of wavelength favorable to the sublimation or vaporization of the matrix. Preferably, an ultraviolet or even infrared wavelength will be used. This ionization may, for example, be carried out with a nitrogen laser emitting a UV beam at 337 nm (for example used in the VITEK® MS system), or with a neodymium-doped yttrium lithium fluoride (Nd:YLF) laser emitting at 349 nm, for example used in the VITEK® MS PRIME system).

The time of flight taken for the ions to reach the detector is used to calculate their mass. Thus, a mass spectrum is obtained, representing the intensity of the signal corresponding to the number of ionized molecules of a given mass-to-charge [m/z], as a function of the m/z ratio of the molecules striking the detector. The mass-to-charge ratio [m/z] is expressed in thomsons (Th).

In particular, the mass spectrum is generated by virtue of the detection of at least a portion of the molecules that have been ionized and accelerated and then allowed to move freely in a tube under reduced pressure, so as to measure the time that they take to travel through the tube under reduced pressure and to obtain a signal corresponding to the number of ionized molecules reaching the detector of the spectrometer at a given instant. A non-calibrated spectrum, comprising on the x-axis the time of flight of the ions and on the y-axis the intensity of the observed signal, is thus obtained. It is converted into a mass spectrum by calculating the mass-to-charge ratios (m/z) corresponding to the times of flight of the detected molecules. To do this, a time-of-flight calibration equation is established using reference molecules. This results in a calibrated mass spectrum corresponding to the signal of the number of ionized molecules of a given mass-to-charge m/z, as a function of the m/z ratio of the molecules detected.

During the calibration step, the peaks corresponding to the so-called reference molecules, in ionized form (called ion of a reference molecule) of the calibration composition, referred to as reference peaks, are identified and their theoretical masses-to-charge m/z will be used as calibration values. This identification can be carried out by comparing the measured times of flight, called the experimental times of flight, of the peaks of the uncalibrated spectrum with the theoretical masses of said ions of the reference proteins, or else with the measured masses-to-charge m/z, referred to as the experimental masses-to-charge m/z, of the mass spectrum if a previous calibration is already available in the instrument and applied. Once these calibration values have been identified, a calibration relationship and an error are calculated. Various linear or nonlinear regression methods may be used to calculate such a calibration relationship. By way of example, mention may be made of the least squares method or any method well known to a person skilled in the art, for performing a calibration. In general, mass spectrometers are equipped with suitable software for implementing such methods. The calculation of an error in parts per million (ppm) is carried out for each reference mass-to-charge value (or reference protein ion), by considering the corresponding experimental value obtained and theoretical value. To do this, the following formula can be: (theoretical mass-experimental mass)/theoretical mass*10⁶. For the set of the ions of the selected reference proteins, it is then possible to calculate a mean error which corresponds to the mean value of the errors expressed in absolute values. The lower the mean error, the better the calibration.

The calibration relationship thus established is then applied to determine the masses-to-charge of the peaks of ions of proteins or molecules present in the analyzed samples, on characterization zones of the MALDI plate. These corrected masses, also called calibrated masses, are obtained in step iii) of the method for characterizing a sample, as defined above.

In summary, the calibration compositions and the method for calibrating a mass spectrometer according to the invention make it possible to obtain precise measurements of the masses of the ions analyzed, and therefore a reliable identification of microorganisms. The calibration compositions and the calibration method according to the invention are simple to implement and can be used in various analytical applications, and in particular for the identification of microorganisms, by mass spectrometry using the MALDI technique, and in particular the MALDI-TOF technique. Within the context of the invention, the MALDI-TOF analysis may be a simple MALDI-TOF analysis, or alternatively a MALDI-TOF-TOF analysis.

The methods according to the invention (calibration method and method for characterizing a sample) are computerized or automated methods. In the methods according to the invention, the steps of acquisition, calculation, comparison, determination, etc., are carried out by a computer or an electronic device programmed to execute said steps, integrated into the spectrometer used. Specific software will be able to be developed and installed on the computer or the electronic device which will be used to execute the calibration method and, in particular, to obtain the calibration relationship. In particular, the software is programmed to take care of the required functionalities, such as the acquisition of the mass spectrometry data, the calculation of the masses-to-charge of the mass peaks in the mass spectrum corresponding to ions of unknown molecules or proteins.

For the identification, on the mass spectrum of the calibration composition, of the peaks corresponding to the calibration values, the calibration software can be programmed to analyze the mass spectrometry data and identify the peaks corresponding to the calibration values. This step may involve adaptive peak detection, mass-to-charge comparison, and thresholding algorithms in order to accurately identify relevant peaks.

For the calculation of the calibration relationship, once the peaks corresponding to the calibration values have been identified, the calibration software performs a calculation to determine the calibration relationship. This calculation may use different statistical methods, such as linear or nonlinear regression, in order to establish a mathematical equation obtained from the theoretical masses-to-charge of the ions of the reference proteins and the masses-to-charge measured for the peaks corresponding to said ionized reference proteins, used as calibration values.

For the application of the calibration relationship, once this has been determined, the software applies it in order to convert the measured (also referred to as experimental) times of flight or masses-to-charge of the peaks corresponding to an unknown entity on the mass spectra obtained for the samples to be analyzed into calibrated masses-to-charge, which are then plotted on the generated mass spectrum. This step makes it possible to obtain accurate measurements of the masses-to-charge of the ions present in the analyzed samples.

The samples analyzed may be of varying origins. As examples, mention may be made of samples of biological origin, in particular animal or human. Such a sample may correspond to a sample of biological fluid, such as whole blood, serum, plasma, urine, cerebrospinal fluid, organic secretion, or to a tissue swab or isolated cells. This sample will be able to be deposited as it is, or will preferably be subjected, prior to deposition on the MALDI plate, to a preparation of enrichment or culture type, to a concentration and/or to a step of extraction or purification according to methods known to a person skilled in the art. The sample may also be an agri-food product such as meat, milk, yoghurt, and any other consumable product that might be contaminated, or a cosmetic or pharmaceutical product. Here again, it will be possible to subject such a product to a preparation of enrichment or culture type, to a concentration and/or to a step of extraction or purification, before being deposited on the MALDI plate. The sample to be analyzed will usually come from a culture in a broth or on an agar plate for enriching it in microorganisms to be searched. For example, it is possible to directly deposit a biomass, a drop of a suspension of microorganisms, in ultrapure water or a buffer.

The examples below, with reference to some of the appended figures, illustrate the invention, but are in no way limiting.

In the examples below, the optical density is measured with the DENSIMAT densitometer (bioMérieux, France) following the recommendations in the user manual (reference 99535, version C, published in 2007).

### EXAMPLE 2: EVALUATION OF THE STABILITY OF A SUSPENSION OF E. COLI IN A COMMERCIAL SOLVENT MIXTURE (BIOMÉRIEUX; REF. 411071)

The E. coli strain ATCC 8739, commercially available and purchased from the ATCC, was added until a concentration of 5 McF (corresponding to an optical density at 550 nm of 1.0-1.1) was obtained in an α-cyano-4-hydroxycinnamic acid matrix (referred to as HCCA matrix) composed of a mixture of 333 ml of ethanol, 333 ml of acetonitrile, 333 ml of water, 30 ml of trifluoroacetic acid and 31 g of HCCA matrix (bioMérieux; ref. 411071). The HCCA matrix is commonly used in MALDI-TOF mass spectrometers for the measurement of peptides and proteins.

The stability of the suspension was then analyzed with a MALDI-TOF mass spectrometer (VITEK® MS, bioMérieux), following the evolution of the calibration peaks between TO and T7 days (7 days after TO). A gradual change in the shape of the peaks was observed over all of the spectra.

For example, the doubly charged peak characteristic of the DNA binding protein HU-alpha was clearly visible at T0 (m/z 4767). However, a third peak had appeared at T24h (1 day after T0), a fourth at T48h (2 days after T0), and 8 peaks were detected at T7d (7 days after T0) (FIG. 2).

Degradation was thus observed over time and, given the mixture of solvents used (ethanol, acetonitrile and trifluoroacetic acid), the degradation is of chemical rather than biological origin. Considering that the observed delta of the masses-to-charge of the ionized proteins between the different peaks over time was approximately 28 Da and that the presence of ethanol and trifluoroacetic acid can protonate the proteins, the inventors believe that it is likely that the degradation corresponds to ethylation.

Consequently, the mixture of solvents in the HCCA matrix commonly used in MALDI-TOF instruments does not make it possible to achieve sufficient stability of the calibration peaks of an E. coli strain over time, and therefore cannot be used as a solvent in the calibration solution.

### EXAMPLE 3: ABSENCE OF IMPACT OF THE METHOD OF CULTURING THE STRAIN ON THE PERFORMANCE OF A CALIBRATION COMPOSITION ACCORDING TO THE INVENTION

Two calibration compositions according to the invention were prepared from the ATCC 8739 E. coli strain used in example 2, but cultured according to two different methods.
A. Protocol for preparing a calibration composition from the ATCC 8739 E. coli strain obtained by growth on a Petri dish in solid agar medium:
culturing the strain on Petri dishes in solid agar medium (bioMérieux; ref. 43039) at 37 °C for 18 to 24 hours,
diluting the strain to 5 McF (i.e. to obtain an optical density at 550 nm of 1.0-1.1) in 70% (v/v) ethanol (i.e. 70% by volume ethanol and 30% by volume water), so as to obtain a calibration composition,
filling tubes (50 μL, 100 μL and 200 μL) with said composition,
storing the tubes: ambient temperature (18-24 °C) and 2-8 °C.

When using the calibration composition, 1 μL of said composition and 1 μL of HCCA matrix are deposited on the MALDI plate.
B. Protocol for preparing a calibration composition from the same ATCC 8739 E. coli strain obtained by broth culture:
culturing the strain in trypticase soy culture broth (bioMérieux; ref. 42100) at 37 °C for 18 to 24 hours,
centrifuging for 10 minutes at 4500 rpm so as to concentrate the cells in a pellet,
doubly rinsing the pellet with water,
resuspending the pellet in a 70% ethanol solution and diluting in order to obtain a calibration composition according to the invention containing E. coli bacteria concentrated at 5 McF (which is equivalent to an optical density at 550 nm of 1.0-1.1) in 70% ethanol,
filling tubes (50 μL, 100 μL and 200 μL) with said composition,
storing the tubes: at ambient temperature (18-24 °C) or at 2-8 °C.

When using the calibration composition, 1 μL of said composition and 1 μL of HCCA matrix are deposited on the MALDI plate.

The calibration compositions prepared according to the two preceding protocols were then used to calibrate a MALDI-TOF mass spectrometer (VITEK® MS, bioMérieux). Both compositions made it possible to obtain a very good calibration of the mass spectrometer, because the reference peaks (including in particular the calibration values used during the calibration) were always present and not degraded (data not presented), thus demonstrating that the method for producing the strain (culture on Petri dish or in culture broth) does not affect the quality of the calibration.

Moreover, after 5 months of storage at ambient temperature, no degradation was observed on the peaks of the various spectra (FIG. 3). In other words, the peaks observed in FIG. 3 show the expected masses for the ATCC 8739 strain, as shown in table 2. In contrast to the formulation of example 2, no degradation is observed, and the masses of the peaks remain constant to within analytical error. This analytical error is inherent to the MALDI-TOF technology. The results obtained with storage between 2-8 °C were also similar.

Consequently, the method of culturing the E. coli strains used to obtain the calibration composition does not affect either the quality of the calibration on a MALDI-TOF mass spectrometer, or even the stability over time of said composition.

### EXAMPLE 4: EVALUATION OF THE IMPACT OF EVAPORATION ON THE QUALITY OF A CALIBRATION COMPOSITION ACCORDING TO THE INVENTION

Calibration composition used: E. coli ATCC 8739 strain suspended at a concentration of 5 McF (i.e. to obtain an optical density at 550 nm of 1.0-1.1) in a 70% ethanol solution and stored in 50 μL, 100 μL, 200 μL and 300 μL tubes.

The tubes were left open at ambient temperature (24 °C±1 °C) until 20%, 50% or 80% of the initial volume had evaporated. The various partially evaporated calibration compositions were then used as calibration solution in a MALDI-TOF mass spectrometer (VITEK® MS, bioMérieux).

After analysis, all of the tested compositions made it possible to obtain a very good calibration of the mass spectrometer regardless of the initial volume and the degree of solvent evaporation. The various MALDI-TOF spectra for the 100 μL tube with an evaporation of 20%, 50% or 80% are presented in FIG. 4. Aside from the analytical error inherent to the MALDI-TOF technology, the spectra are very similar: the peaks described in table 2 are observed with their expected mass; they therefore make it possible to calibrate the spectrum.

### EXAMPLE 5: PREPARATION OF A CALIBRATION COMPOSITION ACCORDING TO THE INVENTION OF E. COLI BL21 IN 70% ETHANOL

Similar to example 3 and using preparation protocol A, a calibration composition according to the invention was prepared with the E. coli strain BL21 (E. coli BL21 suspended in 70% v/v ethanol).

As demonstrated in example 6 below, this solution allowed excellent calibration results to be obtained on a MALDI-TOF mass spectrometer.

### EXAMPLE 6: CALIBRATION OF THE MALDI-TOF INSTRUMENT USING A CALIBRATION COMPOSITION ACCORDING TO THE INVENTION

In general, a MALDI-TOF mass spectrometer measures the time of flight of ions in a vacuum, after they have been accelerated by an electric field. The time of flight is related to the mass of the ions according to the law of kinetic energy:
E_{c}=½mV² with E_{c} the kinetic energy, m the mass and V the velocity of the ions.

It is therefore possible to determine the mass of the ions from the measurement of their time of flight by establishing the relationship linking these two physical quantities, generally by means of a quadratic equation, i.e. a polynomial of degree 2.

In some cases, a polynomial of higher degree may be used to account for physical and electronic imperfections of the mass spectrometer. In practice, the equation is calibrated using molecules of known masses in order to take into account the experimental variations inherent to the instrument, to the MALDI plate on which the sample was deposited or to the acquisition conditions (temperature, pressure in the vacuum tube, etc.).

The instrument may be delivered by the manufacturer with a pre-saved factory calibration. This allows the user to acquire and analyze the first spectra using thomson (Th) values directly. The thomson is a unit corresponding to the mass divided by the charge of the ion. Thereafter, the instrument can be regularly recalibrated using Th spectra without ever resorting to time-of-flight values. Thus, the user becomes accustomed to adjusting the instrument and visualizing the spectra using masses and not times of flight.

The calibration quality can easily be evaluated by calculating the mean of the errors for the masses-to-charge of the ions of the reference proteins. As a reminder, this mean of the errors is calculated using the absolute values of said errors.

By way of example, a MALDI-TOF mass spectrum obtained with a VITEK® MS spectrometer from bioMérieux was calibrated using the calibration composition prepared according to example 5.

The acquisition range of the instrument was set between 2000 and 20 000 Th, the usual mass-to-charge range for identifying microorganisms in microbiology laboratories. The spectrum of the calibration composition obtained exhibiting the uncalibrated experimental m/z values is shown in FIG. 5.

This spectrum was then calibrated using a set of masses-to-charge of protein ions present in E. coli extracted from table 2. In table 2, in the column “charge of the ion”, s denotes an ion bearing one charge, d denotes an ion bearing two charges. The proteins exhibit post-translational modifications with respect to the protein whose name and sequence is given. In the column “post-translational modifications”, c corresponds to the cleavage of methionine at the N-terminal and m corresponds, after cleavage of methionine at the N-terminal, to a methylation of the 2^{nd} amino acid (which is then terminal). The name of the ion of the reference protein is given such as to combine the name of the protein corresponding to the NCBI sequence, its charge and the post-translational modification(s).

TABLE 2
32 theoretical m/z selected for E. coli ATCC 8739

| | Reference | Post-translational | Charge of | Ionized | |
|---|---|---|---|---|---|
| NCBI number | protein | modifications | the ion | protein | m/z (Th) |
| WP_001051798.1 | l33 | mc | d | l33.dmc | 3128.18 |
| WP_000290727.1 | l32 | c | d | l32.dc | 3158.57 |
| WP_001140433.1 | l30 | c | d | l30.dc | 3206.28 |
| WP_001124225.1 | l35 | c | d | l35.dc | 3579.84 |
| WP_000644741.1 | l29 | | d | l29.d | 3637.70 |
| WP_001030593.1 | CsbD | | d | CsbD.d | 4163.59 |
| WP_001144069.1 | bs21c | c | d | bs21.dc | 4185.35 |
| WP_000868187.1 | bl36 | | s | bl36.s | 4365.31 |
| WP_000091955.1 | bl28 | c | d | bl28.dc | 4438.61 |
| WP_001043542.1 | dna1 | | d | dna1.d | 4613.75 |
| WP_001044513.1 | dna2 | | d | dna2.d | 4768.45 |
| WP_001274021.1 | bs20 | c | d | bs20.dc | 4777.56 |
| WP_000059466.1 | us15 | c | d | us15.dc | 5069.74 |
| WP_000841554.1 | s14 | | s | s14.s | 5096.78 |
| WP_001138117.1 | us19 | c | d | us19.dc | 5150.50 |
| WP_000831330.1 | bl34 | | s | bl34.s | 5381.35 |
| WP_001229265.1 | ihf | c | d | ihf.dc | 5612.32 |
| WP_001051798.1 | l33 | mc | s | l33.smc | 6255.36 |
| WP_000290727.1 | bl32 | c | s | bl32.sc | 6316.14 |
| WP_001140433.1 | l30 | c | s | l30.sc | 6411.55 |
| WP_001124225.1 | bl35 | c | s | bl35.sc | 7158.68 |
| WP_000644741.1 | ul29 | | s | ul29.s | 7274.39 |
| WP_001144069.1 | bs21 | c | s | bs21.sc | 8369.69 |
| WP_000091955.1 | bl28c | c | s | bl28.sc | 8876.22 |
| WP_000940595.1 | l27 | c | s | l27.sc | 8994.20 |
| WP_001043542.1 | dna1 | | s | dna1.s | 9226.49 |
| WP_001044513.1 | dna2 | | s | dna2.s | 9535.89 |
| WP_001274021.1 | bs20 | c | s | bs20.sc | 9554.12 |
| WP_000059466.1 | us15 | c | s | us15.sc | 10138.48 |
| WP_001138117.1 | us19 | c | s | us19.sc | 10300.00 |
| WP_001229265.1 | ihf | c | s | ihf.sc | 11223.63 |
| WP_001118930.1 | s14 | c | s | s14.sc | 11450.19 |

The ions of the reference proteins are searched with a tolerance of 800 ppm to calibrate the spectrum by the method of least squares using a quadratic equation.

The mean of the errors before calibration is 372.69 ppm. This mean value is then greatly reduced to 68.50 ppm after the first calibration, thus demonstrating that the masses-to-charge recalibrated by the composition according to the invention (“after 1st calibration” columns, table 3) are more accurate than before calibration.

In order to improve the calibration a little further, it is even possible to perform a second calibration after reducing the tolerance to 300 ppm. This operation eliminates some erroneous associations that are liable to slightly distort the first calibration. These erroneous associations correspond to peaks erroneously attributed to a given theoretical mass, taking into account a large error tolerance (800 ppm). This second calibration slightly improves the measurement accuracy by considering only peaks with an error of less than 300 ppm. The mean of the errors is then equal to 63.18 ppm.

Interestingly, it is not necessary to find all the masses-to-charge searched on the mass spectrum. For instance, peak 28 is not observed in the spectrum before recalibration. After the first recalibration, peaks 23 and 27 exceed the tolerances and are no longer used for the 2nd calibration. It is therefore not necessary for all of the peaks to be present to ensure a good calibration. The method is resilient to the absence of one or more peaks. This property is particularly important for calibrating a MALDI-TOF spectrum. This is because the MALDI-TOF technology has a stochastic behavior that can randomly cause the disappearance or mass shift of certain peaks. For a given sample, certain peaks may be visible or invisible from one acquisition to another, depending on the crystals sublimated by the laser beam. Some peaks may also have a more or less noisy shape, which may lead to a more or less significant error in the detected mass.

TABLE 3
m/z readings for a calibration composition according to the invention (E. coli BL21; 70% v/v ethanol),
before and after calibration, using the 32 theoretical m/z of E. coli ATCC 8739 shown in table 2.

| | | | Intensity of | before calibration | | after 1st calibration | | after 2nd calibration | |
|---|---|---|---|---|---|---|---|---|---|
| | Reference | Theoretical | peaks observed | Experimental | Error | Experimental | Error | Experimental | Error |
| Peak | protein | m/z (Th) | (AU) | m/z (Th) | (ppm) | m/z (Th) | (ppm) | m/z (Th) | (ppm) |
| 1 | l33.dmc | 3128.18 | 9 137 994.00 | 3 126.50 | −537.34 | 3 128.10 | −26.44 | 3 128.17 | −2.97 |
| 2 | l32.dc | 3158.57 | 8 659 036.00 | 3 156.75 | −576.54 | 3 158.35 | −68.92 | 3 158.42 | −46.65 |
| 3 | l30.dc | 3206.28 | 3 152 315.00 | 3 204.43 | −577.33 | 3 206.04 | −74.79 | 3 206.11 | −54.34 |
| 4 | l35.dc | 3579.84 | 12 966 253.00 | 3 578.02 | −508.66 | 3 579.69 | −41.19 | 3 579.72 | −32.67 |
| 5 | l29.d | 3637.70 | 13 286 487.00 | 3 635.91 | −492.31 | 3 637.59 | −29.62 | 3 637.62 | −22.62 |
| 6 | CsbD.d | 4163.59 | 3 788 764.75 | 4 161.68 | −458.95 | 4 163.45 | −33.32 | 4 163.44 | −37.08 |
| 7 | bs21.dc | 4185.35 | 532 103.94 | 4 182.98 | −566.58 | 4 184.75 | −142.20 | 4 184.74 | −146.30 |
| 8 | bl36.s | 4365.31 | 26 988 438.00 | 4 363.09 | −508.81 | 4 364.90 | −94.90 | 4 364.87 | −101.58 |
| 9 | bl28.dc | 4438.61 | 9 653 424.00 | 4 436.87 | −392.17 | 4 438.69 | 17.67 | 4 438.65 | 10.06 |
| 10 | dna1.d | 4613.75 | 11 180 064.00 | 4 611.91 | −398.97 | 4 613.76 | 1.87 | 4 613.71 | −7.69 |
| 11 | dna2.d | 4768.45 | 10 108 585.00 | 4 767.02 | −299.98 | 4 768.90 | 93.41 | 4 768.84 | 82.42 |
| 12 | bs20.dc | 4777.56 | 4 900 148.00 | 4 775.93 | −341.29 | 4 777.81 | 51.70 | 4 777.75 | 40.63 |
| 13 | us15.dc | 5069.74 | 3 621 066.00 | 5 068.28 | −288.07 | 5 070.21 | 92.31 | 5 070.14 | 79.18 |
| 14 | s14.s | 5096.78 | 25 888 934.00 | 5 094.79 | −390.60 | 5 096.72 | −11.25 | 5 096.66 | −24.52 |
| 15 | us19.dc | 5150.50 | 9 259 382.00 | 5 149.29 | −234.98 | 5 151.23 | 142.14 | 5 151.16 | 128.59 |
| 16 | bl34.s | 5381.35 | 31 862 212.00 | 5 379.65 | −316.01 | 5 381.63 | 52.54 | 5 381.55 | 38.02 |
| 17 | ihf.dc | 5612.32 | 2 463 163.00 | 5 611.12 | −213.86 | 5 613.14 | 146.76 | 5 613.06 | 131.64 |
| 18 | l33.smc | 6255.36 | 22 558 522.00 | 6 253.33 | −324.63 | 6 255.47 | 17.42 | 6 255.37 | 2.27 |
| 19 | bl32.sc | 6316.14 | 12 393 123.00 | 6 314.41 | −273.98 | 6 316.56 | 66.50 | 6 316.46 | 51.44 |
| 20 | l30.sc | 6411.55 | 5 105 760.00 | 6 409.83 | −268.34 | 6 412.00 | 69.77 | 6 411.90 | 54.91 |
| 21 | bl35.sc | 7158.68 | 28 049 696.00 | 7 155.89 | −389.89 | 7 158.19 | −67.84 | 7 158.11 | −80.05 |
| 22 | ul29.s | 7274.39 | 22 238 468.00 | 7 272.52 | −257.13 | 7 274.85 | 62.68 | 7 274.76 | 51.06 |
| 23 | bs21.sc | 8369.69 | 184 524.66 | 8 364.10 | −668.33 | NA | NA | NA | NA |
| 24 | bl28.sc | 8876.22 | 11 786 749.00 | 8 872.43 | −427.17 | 8 875.06 | −130.71 | 8 875.06 | −131.14 |
| 25 | l27.sc | 8994.20 | 283 529.88 | 8 990.59 | −401.53 | 8 993.24 | −106.43 | 8 993.25 | −105.83 |
| 26 | dna1.s | 9226.49 | 24 487 758.00 | 9 223.63 | −310.07 | 9 226.33 | −17.52 | 9 226.35 | −14.86 |
| 27 | dna2.s | 9535.89 | 67 042 984.00 | 9 536.21 | 33.56 | NA | NA | NA | NA |
| 28 | bs20.sc | 9554.12 | NA | NA | NA | NA | NA | NA | NA |
| 29 | us15.sc | 10138.48 | 4 802 329.50 | 10 135.61 | −283.16 | 10 138.49 | 0.87 | 10 138.60 | 12.29 |
| 30 | us19.sc | 10300.00 | 9 148 744.00 | 10 297.59 | −234.04 | 10 300.50 | 48.67 | 10 300.64 | 61.74 |
| 31 | ihf.sc | 11223.63 | 4 186 346.25 | 11 218.82 | −428.74 | 11 221.92 | −152.51 | 11 222.18 | −129.57 |
| 32 | s14.sc | 11450.19 | 582 660.50 | 11 448.47 | −150.24 | 11 451.62 | 124.51 | 11 451.91 | 150.02 |

| | | | |
|---|---|---|---|
| Mean of the errors (ppm) | 372.69 | 68.50 | 63.18 |

### EXAMPLE 7: STABILITY STUDY OF A CALIBRATION COMPOSITION ACCORDING TO EXAMPLE 5

The possibility of calibration via the calibration composition according to example 5, which is stable over time, was established by verifying that it was possible to calibrate a VITEK® MS MALDI-TOF mass spectrometer with a batch of this calibration composition freshly prepared according to example 5 (condition T0), then with the same batch of calibration composition stored for 9 months (condition T9) or 12 months (condition T12) at 2-8 °C.

In order to simulate intensive use conditions, batches stored at 2-8 °C for 9 or 12 months were then left unstoppered for 1 h30 in order to allow some of the solvent to evaporate before then being closed again for 180 h at ambient temperature (18-25 °C).

The masses-to-charge before and after calibration, obtained with the list of 32 reference peaks are shown in tables 4, 4bis and 4ter below. The measurement errors for the calibration peaks have been plotted in FIG. 6 (A: before calibration; B: after calibration).

An excellent calibration is obtained at TO, T9 and T12, characterized by a significant reduction in the mean of the errors, going from 157.69 to 17.64 ppm, from 186.45 to 19.45 ppm and from 287.29 to 29.01 ppm, respectively.

These results demonstrate an excellent calibration with a liquid solution from T0 to 12 months despite the simulation of intensive use with an evaporation time of 1 h30 and a duration of 7.5 days at ambient temperature. Interestingly, the improvement in the calibration quality is obtained even if the presence of some peaks/values (denoted NA) is not observed.

TABLE 4
m/z readings for a calibration composition according to the invention
(E. coli BL21; 70% v/v ethanol), freshly prepared, before and after calibration
with reference to the 32 theoretical m/z of E. coli ATCC 8739.

| | | | before calibration | | after calibration | |
|---|---|---|---|---|---|---|
| Reference | Reference | Theoretical | Experimental | error | Experimental | error |
| peak | protein | m/z (Th) | m/z (Th) | (ppm) | m/z (Th) | (ppm) |
| 1 | l33.dmc | 3128.18 | 3126.60788 | 502.57 | 3128.1546 | 8.12 |
| 2 | l32.dc | 3158.57 | 3156.92742 | 520.04 | 3158.4602 | 34.76 |
| 3 | l30.dc | 3206.28 | 3204.66313 | 504.28 | 3206.1739 | 33.09 |
| 4 | l35.dc | 3579.84 | 3578.55434 | 359.14 | 3579.8966 | −15.81 |
| 5 | l29.d | 3637.70 | 3636.37196 | 365.08 | 3637.6887 | 3.11 |
| 6 | CsbD.d | 4163.59 | 4162.56905 | 245.21 | 4163.6612 | −17.10 |
| 7 | bs21.dc | 4185.35 | 4184.13261 | 290.87 | 4185.2158 | 32.06 |
| 8 | bl36.s | 4365.31 | 4364.25602 | 241.44 | 4365.2655 | 10.19 |
| 9 | bl28.dc | 4438.61 | 4437.80499 | 181.37 | 4438.7849 | −39.40 |
| 10 | dna1.d | 4613.75 | 4612.72008 | 223.23 | 4613.6305 | 25.90 |
| 11 | dna2.d | 4768.45 | 4767.73379 | 150.20 | 4768.5838 | −28.06 |
| 12 | bs20.dc | 4777.56 | 4776.79268 | 160.61 | 4777.6392 | −16.58 |
| 13 | us15.dc | 5069.74 | 5068.98871 | 148.19 | 5069.7246 | 3.04 |
| 14 | s14.s | 5096.78 | 5096.07878 | 137.58 | 5096.8046 | −4.83 |
| 15 | us19.dc | 5150.50 | 5149.94081 | 108.57 | 5150.6467 | −28.48 |
| 16 | bl34.s | 5381.35 | 5380.89282 | 84.96 | 5381.515 | −30.66 |
| 17 | ihf.dc | 5612.32 | 5611.88594 | 77.34 | 5612.4269 | −19.05 |
| 18 | l33.smc | 6255.36 | 6255.01767 | 54.73 | 6255.3458 | 2.27 |
| 19 | bl32.sc | 6316.14 | 6315.74131 | 63.12 | 6316.0504 | 14.19 |
| 20 | l30.sc | 6411.55 | 6411.19433 | 55.47 | 6411.4737 | 11.90 |
| 21 | bl35.sc | 7158.68 | 7158.54554 | 18.78 | 7158.6075 | 10.13 |
| 22 | ul29.s | 7274.39 | 7274.37257 | 2.40 | 7274.4032 | −1.81 |
| 23 | bs21.sc | 8369.69 | 8369.8208 | −15.63 | 8369.5863 | 12.39 |
| 24 | bl28.sc | 8876.22 | 8876.5022 | −31.79 | 8876.1642 | 6.29 |
| 25 | l27.sc | 8994.20 | 8994.26028 | −6.70 | 8993.9 | 33.35 |
| 26 | dna1.s | 9226.49 | 9226.95059 | −49.92 | 9226.5481 | −6.30 |
| 27 | dna2.s | 9535.89 | 9536.68442 | −83.31 | 9536.2298 | −35.63 |
| 28 | bs20.sc | 9554.12 | 9554.7377 | −64.65 | 9554.2801 | −16.76 |
| 29 | us15.sc | 10138.48 | 10138.7553 | −27.16 | 10138.2121 | 26.42 |
| 30 | us19.sc | 10300.00 | 10300.3931 | −38.17 | 10299.8291 | 16.59 |
| 31 | ihf.sc | 11223.63 | 11224.4825 | −75.96 | 11223.8228 | −17.18 |
| 32 | s14.sc | 11450.19 | 11450.9006 | −62.06 | 11450.2236 | −2.93 |

| | | |
|---|---|---|
| Mean of the errors (ppm) | 157.69 | 17.64 |

TABLE 4bis
m/z readings for a calibration composition according to the invention (E. coli
BL21; 70% v/v ethanol), stored for 9 months at 2-8 °C, before and after calibration

| | | | before calibration | | after calibration | |
|---|---|---|---|---|---|---|
| Reference | Reference | | Experimental | Error | Experimental | Error |
| peak | protein | m/z (Th) | m/z (Th) | (ppm) | m/z (Th) | (ppm) |
| 1 | l33.dmc | 3128.18 | 3126.83492 | 429.99 | 3128.2612 | −25.96 |
| 2 | l32.dc | 3158.57 | 3156.98992 | 500.25 | 3158.4007 | 53.60 |
| 3 | l30.dc | 3206.28 | 3204.89772 | 431.12 | 3206.2836 | 1.12 |
| 4 | l35.dc | 3579.84 | 3578.73626 | 308.32 | 3579.9272 | −24.36 |
| 5 | l29.d | 3637.70 | 3636.46898 | 338.41 | 3637.6296 | 19.35 |
| 6 | CsbD.d | 4163.59 | 4162.70566 | 212.40 | 4163.5869 | 0.74 |
| 7 | bs21.dc | 4185.35 | 4184.36727 | 234.80 | 4185.2369 | 27.02 |
| 8 | bl36.s | 4365.31 | 4364.60511 | 161.48 | 4365.3778 | −15.53 |
| 9 | bl28.dc | 4438.61 | 4437.9629 | 145.79 | 4438.6959 | −19.35 |
| 10 | dna1.d | 4613.75 | 4612.99294 | 164.09 | 4613.631 | 25.79 |
| 11 | dna2.d | 4768.45 | 4768.02062 | 90.05 | 4768.5741 | −26.03 |
| 12 | bs20.dc | 4777.56 | 4776.97733 | 121.96 | 4777.5259 | 7.14 |
| 13 | us15.dc | 5069.74 | 5069.30291 | 86.22 | 5069.6908 | 9.70 |
| 14 | s14.s | 5096.78 | 5096.50185 | 54.57 | 5096.8747 | 18.58 |
| 15 | us19.dc | 5150.50 | 5150.26241 | 46.13 | 5150.6055 | −20.48 |
| 16 | bl34.s | 5381.35 | 5381.23563 | 21.25 | 5381.4503 | −18.64 |
| 17 | ihf.dc | 5612.32 | 5612.13847 | 32.35 | 5612.2237 | 17.16 |
| 18 | l33.smc | 6255.36 | 6255.79558 | −69.63 | 6255.515 | −24.78 |
| 19 | bl32.sc | 6316.14 | 6316.40822 | −42.47 | 6316.0928 | 7.47 |
| 20 | l30.sc | 6411.55 | 6411.87204 | −50.23 | 6411.5016 | 7.55 |
| 21 | bl35.sc | 7158.68 | 7159.43407 | −105.34 | 7158.6269 | 7.42 |
| 22 | ul29.s | 7274.39 | 7275.40238 | −139.17 | 7274.5266 | −18.78 |
| 23 | bs21.sc | 8369.69 | 8370.99381 | −155.78 | 8369.4571 | 27.83 |
| 24 | bl28.sc | 8876.22 | 8877.88161 | −187.20 | 8876.0316 | 21.23 |
| 25 | l27.sc | 8994.20 | 8995.79727 | −177.59 | 8993.8737 | 36.28 |
| 26 | dna1.s | 9226.49 | 9228.66462 | −235.69 | 9226.595 | −11.38 |
| 27 | dna2.s | 9535.89 | 9538.59136 | −283.28 | 9536.3259 | 45.71 |
| 28 | bs20.sc | 9554.12 | 9556.50185 | −249.30 | 9554.225 | −10.99 |
| 29 | us15.sc | 10138.48 | 10140.9085 | −239.53 | 10138.2573 | 21.97 |
| 30 | us19.sc | 10300.00 | 10302.8757 | −279.19 | 10300.1196 | −11.61 |
| 31 | ihf.sc | 11223.63 | NA | NA | NA | NA |
| 32 | s14.sc | 11450.19 | NA | NA | NA | NA |

| | | |
|---|---|---|
| Mean of the errors (ppm) | 186.45 | 19.45 |

TABLE 4ter
m/z readings for a calibration composition according to the invention (E. coli BL21;
70% v/v ethanol), stored for 12 months at 2-8 °C, before and after calibration

| | | | before calibration | | after calibration | |
|---|---|---|---|---|---|---|
| Reference | | | Experimental | Error | Experimental | Error |
| peak | Protein | m/z (Th) | m/z (Th) | (ppm) | m/z (Th) | (ppm) |
| 1 | l33.dmc | 3128.18 | 3127.19497 | 314.89 | 3128.1636 | 5.24 |
| 2 | l32.dc | 3158.57 | 3157.50295 | 337.83 | 3158.4777 | 29.22 |
| 3 | l30.dc | 3206.28 | 3205.3046 | 304.22 | 3206.2892 | −2.87 |
| 4 | l35.dc | 3579.84 | 3578.42635 | 394.89 | 3579.4894 | 97.94 |
| 5 | l29.d | 3637.70 | 3636.66451 | 284.65 | 3637.7401 | −11.02 |
| 6 | CsbD.d | 4163.59 | 4162.23983 | 324.28 | 4163.4324 | 37.85 |
| 7 | bs21.dc | 4185.35 | 4184.17767 | 280.10 | 4185.3753 | −6.04 |
| 8 | bl36.s | 4365.31 | 4364.11737 | 273.21 | 4365.3567 | −10.70 |
| 9 | bl28.dc | 4438.61 | 4437.43172 | 265.46 | 4438.6883 | −17.64 |
| 10 | dna1.d | 4613.75 | 4612.25582 | 323.85 | 4613.554 | 42.48 |
| 11 | dna2.d | 4768.45 | 4767.29055 | 243.15 | 4768.6263 | −36.97 |
| 12 | bs20.dc | 4777.56 | 4776.34472 | 254.37 | 4777.6827 | −25.68 |
| 13 | us15.dc | 5069.74 | 5068.28067 | 287.85 | 5069.6909 | 9.68 |
| 14 | s14.s | 5096.78 | 5095.56863 | 237.67 | 5096.9857 | −40.36 |
| 15 | us19.dc | 5150.50 | 5149.2955 | 233.86 | 5150.7262 | −43.92 |
| 16 | bl34.s | 5381.35 | 5380.23178 | 207.80 | 5381.7216 | −69.05 |
| 17 | ihf.dc | 5612.32 | 5610.87708 | 257.10 | 5612.4273 | −19.12 |
| 18 | l33.smc | 6255.36 | 6253.63948 | 275.05 | 6255.3648 | −0.77 |
| 19 | bl32.sc | 6316.14 | 6314.3276 | 286.95 | 6316.07 | 11.08 |
| 20 | l30.sc | 6411.55 | 6409.72456 | 284.71 | 6411.4939 | 8.75 |
| 21 | bl35.sc | 7158.68 | 7156.62849 | 286.58 | 7158.6169 | 8.81 |
| 22 | ul29.s | 7274.39 | 7272.51197 | 258.17 | 7274.5356 | −20.02 |
| 23 | bs21.sc | 8369.69 | 8366.47018 | 384.70 | 8368.8425 | 101.26 |
| 24 | bl28.sc | 8876.22 | 8873.64981 | 289.56 | 8876.1937 | 2.96 |
| 25 | l27.sc | 8994.20 | 8991.6155 | 287.35 | 8994.2002 | −0.02 |
| 26 | dna1.s | 9226.49 | 9223.87473 | 283.45 | 9226.5407 | −5.50 |
| 27 | dna2.s | 9535.89 | 9533.84564 | 214.39 | 9536.6223 | −76.79 |
| 28 | bs20.sc | 9554.12 | 9551.45097 | 279.36 | 9554.2339 | −11.92 |
| 29 | us15.sc | 10138.48 | 10134.9388 | 349.28 | 10137.9366 | 53.60 |
| 30 | us19.sc | 10300.00 | 10296.4694 | 342.78 | 10299.5282 | 45.81 |
| 31 | ihf.sc | 11223.63 | 11220.7281 | 258.56 | 11224.1479 | −46.14 |
| 32 | s14.sc | 11450.19 | NA | NA | NA | NA |

| | | |
|---|---|---|
| Mean of the errors (ppm) | 287.29 | 29.01 |

### EXAMPLE 8: SELECTION OF THE CALIBRATION VALUES ACCORDING TO THE STRAIN USED

As demonstrated in example 6, the calibration suspension prepared with the E. coli strain BL21 makes it possible to improve the calibration during the acquisition of MALDI-TOF spectra. However, the quality of the calibration depends on the reference proteins selected for performing the calibration.

The objective of this example is to present the methodology for selecting, for a given strain (in this case, the E. coli strain BL21), the reference proteins whose theoretical masses-to-charge are known and which will be used for calibration. This methodology is applicable for all other strains of E. coli.

In general, the interpretation of a MALDI-TOF spectrum consists in searching for a list of expected theoretical masses-to-charge from among the experimental values, i.e. the masses-to-charge measured and obtained from the mass spectrum before calibration. This association occurs below a certain accuracy threshold, a tolerance or error denoted in “ppm” for “parts per million”.

### A) Selection of Theoretical Masses-to-Charge for the E. coli Strain BL21

A selection of theoretical masses-to-charge was made over the m/z range extending from 3000 to 12 500 Th (trivially fixed with respect to the capabilities of the mass spectrometer) with the aim of selecting proteins and associated ions in such a way that the corresponding theoretical masses-to-charge are distributed uniformly over said range.

Table 5 below shows the theoretical masses-to-charge (m/z) of a number of pre-selected proteins and the corresponding names, according to the same convention as table 2. Not found in this table 2, the post-translational modifications can also be of the type of formylation or acetylation of the 2^{nd} amino acid after cleavage of the methionine at the N-terminal; these modifications are denoted for a.

TABLE 5
49 theoretical m/z of E. coli BL21

| | | | | Ion of the | |
|---|---|---|---|---|---|
| | ionized | Theoretical | | reference | Theoretical |
| NCBI number | protein | m/z (Th) | NCBI number | protein | m/z (Th) |
| WP_001051798.1 | l33.dmc | 3128.18 | WP_000290727.1 | bl32.sc | 6316.14 |
| WP_000290727.1 | l32.dc | 3158.57 | WP_001140433.1 | l30.sc | 6411.55 |
| WP_000290727.1 | l32.dac | 3179.59 | WP_000828648.1 | us13.s | 6508.45 |
| WP_001140433.1 | l30.dc | 3206.28 | WP_000281320.1 | duf.df | 6547.83 |
| WP_001124225.1 | l35.dc | 3579.84 | WP_001029684.1 | us11.dc | 6857.81 |
| WP_000644741.1 | l29.d | 3637.70 | WP_001124225.1 | bl35.sc | 7158.68 |
| WP_000710769.1 | l31.d | 3936.51 | WP_000644741.1 | ul29.s | 7274.39 |
| WP_001030593.1 | CsbD.d | 4163.59 | WP_000691956.1 | YchN.s | 7707.56 |
| WP_001144069.1 | bs21.dc | 4185.35 | WP_000710769.1 | bl31.s | 7872.02 |
| WP_000868187.1 | bl36.s | 4365.31 | WP_001030593.1 | CsbD.s | 8326.18 |
| WP_000091955.1 | bl28.dc | 4438.61 | WP_001144069.1 | bs21.sc | 8369.69 |
| WP_000135199.1 | bs18.dac | 4449.60 | WP_000091955.1 | bl28.sc | 8876.22 |
| WP_000940595.1 | l27.dc | 4497.60 | WP_000135199.1 | bs18.sac | 8898.19 |
| WP_001043542.1 | dna1.d | 4613.75 | WP_000940595.1 | l27.sc | 8994.20 |
| WP_001044513.1 | dna2.d | 4768.45 | WP_000256450.1 | bs16.sc | 9060.27 |
| WP_001274021.1 | bs20.dc | 4777.56 | WP_001043542.1 | dna1.s | 9226.49 |
| WP_000059466.1 | us15.dc | 5069.74 | WP_001044513.1 | dna2.s | 9535.89 |
| WP_000841554.1 | s14.s | 5096.78 | WP_001274021.1 | bs20.sc | 9554.12 |
| WP_001138117.1 | us19.dc | 5150.50 | WP_000756550.1 | HdeA.s | 9741.83 |
| WP_000831330.1 | bl34.s | 5381.35 | WP_000059466.1 | us15.sc | 10138.48 |
| WP_000031415.1 | YqjD.dc | 5461.10 | WP_001138117.1 | us19.sc | 10300.00 |
| WP_001229265.1 | ihf.dc | 5612.32 | WP_001229265.1 | ihf.sc | 11223.63 |
| WP_001118930.1 | s14.dc | 5725.60 | WP_001118930.1 | s14.sc | 11450.19 |
| WP_001051798.1 | l33.smc | 6255.36 | WP_000447529.1 | l22.s | 12227.19 |
| WP_001276008.1 | ZapA, d | 6298.04 | | | |

### (B) Experimental MALDI-TOF Spectra

In its databases, the applicant has available 14 332 experimental MALDI-TOF spectra of E. coli BL 21, acquired over the course of time (6614 were obtained with the VITEK® MS mass spectrometer and 7718 with the VITEK® MS PRIME mass spectrometer).

Among these spectra, 52 of them allow less than half of the theoretical masses-to-charge searched and are excluded in order to ultimately retain 14 280 experimental MALDI-TOF spectra.

### C) Validation of the Theoretical Masses-to-Charge Used for the Calibration

### C1. First Validation of the Theoretical Masses-to-Charge

Before making use of the interpretation results, the quality of the calibration was checked. This is because a poor calibration can distort the interpretation of experimental peaks, especially as by way of irregular accuracy.

For example, it is possible to have “shifted” theoretical masses-to-charge, systematically adding bias to the calibration, or else “unstable” theoretical masses-to-charge, scrambling the overall accuracy of the calibration. In reality, the two situations collide, hence the need to exclude the theoretical masses that may be disruptive to the calibration.

To do this, only part of the spectra was used. The association between a sample of 7724 MALDI-TOF spectra and the selected 49 theoretical masses resulted in 295 829 interpretations, or about 38 interpretations per spectrum, that is to say 38 observed theoretical, or “experimental”, masses-to-charge out of the 49 searched.

In order to have an overall view of the calibration, the overall interpretation of each theoretical mass-to-charge was displayed on the same graph, which is called the super calibration graph. The median of the interpretation error of each theoretical mass-to-charge was displayed with a variance interval corresponding to the standard deviation of the error, as shown in FIG. 7.

From the super calibration graph in FIG. 7, three criteria then made it possible to exclude the theoretical masses-to-charge that were disruptive to the calibration:
The visibility of the theoretical masses-to-charge: some theoretical masses-to-charge are very infrequently observed and are consequently excluded for the calibration. This applies to four theoretical masses-to-charge.
The mass-to-charge shift: some theoretical masses-to-charge appear to be abnormally shifted, and therefore a threshold of 100 ppm is applied to the median error. This criterion applies to seven theoretical masses-to-charge.
The stability of the mass: some theoretical masses-to-charge allow a higher standard deviation and variance interval. Thus, a threshold of 100 ppm is applied to the standard deviation in order to be able to exclude the theoretical masses-to-charge having a standard deviation of the error that is greater than 100 ppm. This criterion applies to six theoretical masses-to-charge.

By combining the three criteria, twelve distinct theoretical masses-to-charge were excluded for the calibration and are shown in table 6 below.

TABLE 6
12 m/z of a calibration composition according to the invention (E. coli
BL21, 70% v/v ethanol), excluded from the calibration process

| | | | | | Standard |
|---|---|---|---|---|---|
| | | Number of | | Median | deviation |
| | | interpretations | | of the | of the |
| m/z | Ionized | (interpretation | | error | error |
| (Th) | protein | percentage) | | (ppm) | (ppm) |
| 3 179.59 | l32.dac | 7 454 | (97%) | −184.10 | 114.43 |
| 3 936.51 | l31.d | 2 268 | (29%) | −231.79 | 64.31 |
| 5 461.10 | YqjD.dc | 21 | (00%) | 36.62 | 108.98 |
| 5 725.60 | s14.dc | 7 438 | (96%) | 118.39 | 74.39 |
| 6 298.04 | ZapA, d | 3 184 | (41%) | −104.77 | 138.26 |
| 6 508.45 | us13.s | 1 844 | (24%) | 80.27 | 167.31 |
| 6 547.83 | duf.df | 15 | (00%) | 69.66 | 123.52 |
| 7 872.02 | bl31.s | 1 863 | (24%) | −226.12 | 74.06 |
| 8 326.18 | CsbD.s | 7 422 | (96%) | 108.33 | 49.84 |
| 9 060.27 | bs16.sc | 1 | (00%) | −88.11 | — |
| 9 741.83 | HdeA.s | 4 | (00%) | 94.30 | 62.67 |
| 12 227.19 | l22.s | 1 244 | (16%) | −137.60 | 121.97 |

At the end of the first validation, 37 theoretical masses-to-charge were retained.

### C2. Second Validation of the Theoretical Masses-to-Charge

The analysis was then carried out on a sample of 7650 spectra in order to obtain the super calibration graph showing, for each theoretical mass-to-charge, the median of the interpretation error with a variance interval corresponding to the standard deviation of the error.

Then, a threshold of 40 ppm was applied to the median of the error. The application of this threshold allowed 5 theoretical masses-to-charge to be excluded for the calibration, these being listed in table 7 below.

TABLE 7
5 m/z of a calibration composition according to
the invention (E. coli BL21, 70% v/v ethanol),
likewise excluded from the calibration process

| | | | | Standard |
|---|---|---|---|---|
| | | Number of | Median | deviation |
| | | interpretations | of the | of the |
| m/z | Ionized | (interpretation | error | error |
| (Th) | protein | percentage) | (ppm) | (ppm) |
| 4 449.60 | bs18.dac | 6 434 (84%) | −54.84 | 53.81 |
| 4 497.60 | l27.dc | 7 372 (96%) | −39.63 | 39.05 |
| 6 857.81 | us11.dc | 6 231 (81%) | −89.58 | 53.22 |
| 7 707.56 | YchN.s | 3 903 (51%) | −66.21 | 95.75 |
| 8898.19 | bs18.sac | 5 393 (70%) | −69.08 | 49.96 |

At the end of the second validation, 32 theoretical masses-to-charge are retained for the calibration and the 17 not retained are nevertheless searched in order to improve the control. This is summarized in the next section.

### D) Final Interpretation Method

6563 spectra acquired with the VITEK® MS spectrometer were studied.

Before interpretation, the spectra were calibrated using 32 preselected theoretical masses. The calibration consisted in two successive quadratic regressions at the tolerance thresholds of 500 ppm and 300 ppm respectively using the 32 selected calibration theoretical masses-to-charge.

Lastly, a total of 49 (theoretical calibration and additional) masses-to-charge were searched with a tolerance threshold of 300 ppm.

### E) Results

As mentioned above, in order to understand the calibration from a general point of view, the overall interpretation of each theoretical mass-to-charge was displayed on the same graph. The median of the interpretation error of each theoretical mass-to-charge was displayed with a confidence interval corresponding to the standard deviation of the error, as shown in FIG. 8.

Then, the interpretation performance was calculated over the set of the 32 masses-to-charge used as calibration values and the 17 additional masses-to-charge. The results are presented in table 8 below.

TABLE 8
Calibration performance according to the invention

| Set | Mean visibility rate |
|---|---|
| 32 calibration masses | 95% |
| 17 additional masses | 50% |

| | | | Standard |
|---|---|---|---|
| | Median | Mean | deviation |
| | of the | of the | of the |
| | error | errors, | error, |
| | before | before | before |
| Set | calibration | calibration | calibration |
| 32 calibration masses | 119.48 ppm | 152.48 ppm | 126.39 ppm |
| 17 additional masses | (not used) | (not used) | (not used) |

| | | | Standard |
|---|---|---|---|
| | Median | Mean | deviation |
| | of the | of the | of the |
| | error, | errors, | error, |
| | after | after | after |
| Set | calibration | calibration | calibration |
| 32 calibration masses | 19.41 ppm | 25.23 ppm | 25.50 ppm |
| 17 additional masses | 85.60 ppm | 107.62 ppm | 74.85 ppm |

It is apparent from table 8 that, firstly, the 32 masses-to-charge used corresponding to the calibration values are very frequently visible, with a mean visibility rate of 95% over all of the spectra.

Secondly, the absolute measurement error was also corrected by the calibration, with a median decreasing from 119.48 ppm to 19.41 ppm, a mean decreasing from 152.48 ppm to 25.23 ppm and a standard deviation decreasing from 126.39 ppm to 23.50 ppm. In other words, the method for validating the masses-to-charge corresponding to the calibration values made it possible to provide interpretations that were 6 times more accurate, with a measurement uncertainty that was 4 times smaller. The method for selecting the reference theoretical masses-to-charge thus made it possible to significantly improve the calibration performance.

In addition, the mass spectrometer thus calibrated could then be successfully used for the identification of different microorganisms.

### EXAMPLE 9: STABILITY AND PERFORMANCE OF CALIBRATION COMPOSITIONS ACCORDING TO THE INVENTION

Table 9 below provides, by way of indication, the results of a test of stability over time carried out on various calibration compositions according to the invention.

The compositions tested were prepared with E. coli BL 21 bacteria suspended in a pure alcohol solution or aqueous alcohol or acetonitrile solutions, here:
a 100% (v/v) ethanol solution,
a 20% (v/v) ethanol solution,
a 50% (v/v) ethanol solution,
a 70% (v/v) ethanol solution,
a 70% (v/v) isopropanol solution, and
a 70% (v/v) acetonitrile solution.

The stability of these calibration compositions was evaluated according to the calibration performance obtained. This is expressed as mean of the errors associated with the 32 calibration masses selected in example 8.

The measurements were carried out with calibration compositions “freshly” prepared (T0) or after 8 days of storage at ambient temperature (T8).

TABLE 9
Stability of calibration compositions, in terms of calibration
performance (expressed as mean of the errors).

| | before | | before | |
|---|---|---|---|---|
| Calibration | calibration | calibration | calibration | calibration |
| composition | at T0 | at T0 | at T8 | at T8 |
| 20% ETOH | 246.5 | 27.6 | 295.5 | 21.1 |
| 50% ETOH | 132.63 | 29.91 | 118.7 | 19.2 |
| 70% ETOH | 165.8 | 34.7 | 356.1 | 16.2 |
| 100% ETOH | 400.8 | 72.1 | 291.2 | 34.5 |
| 70% ISO | 199.9 | 32.1 | 213.6 | 25.0 |
| 70% ACN | 199.8 | 25.2 | 131.7 | 21.0 |

The six calibration compositions, the calibration performance of which is reported in table 9, therefore make it possible to reduce the mean of the errors by an average factor of approximately 6 at T0 and approximately 10 at T8, which demonstrates an excellent calibration according to the invention.

By way of comparison, calibrations performed with freshly cultured E. coli ATCC 8739 bacteria, as prescribed in the VITEK® MS user manual, obtain means of the errors of the order of 30 ppm after calibration (and according to recent measurements carried out under the same conditions as example 8:109.94 ppm before calibration and 26.9 ppm after calibration).

It should also be noted that a preparation of E. coli BL 21 bacteria, suspended in a 70% formic acid solution, resulted in mass spectra comprising too few peaks to allow calibration.

1. A calibration composition for a mass spectrometer comprising whole E. coli bacteria suspended in a liquid which is based on alcohol(s) and/or on acetonitrile [and is asepticizing, volatile and non-denaturing/non-reactive with regard to the bacterial proteins].

2. The calibration composition as claimed in claim 1, wherein the liquid is based on alcohol(s).

3. The calibration composition as claimed in claim 2, wherein the liquid is an aqueous liquid having a content of alcohol(s) of at least 20% v/v.

4. The calibration composition as claimed in claim 2, wherein the alcohol(s) is/are ethanol and/or isopropanol.

5. The calibration composition as claimed in claim 3, wherein the liquid is a water/alcohol mixture, having an alcohol content of 70% v/v.

6. The calibration composition as claimed in claim 1, wherein the liquid is an aqueous water/acetonitrile mixture.

7. The composition as claimed in claim 1, wherein the E. coli bacteria are of a single strain.

8. The calibration composition as claimed in claim 1, wherein the E. coli bacteria are of the BL21 strain or of the ATCC 8739 strain.

9. The calibration composition as claimed in claim 1, wherein the E. coli bacteria are present at a concentration of from 6×10⁸ to 21×10⁸ cfu/mL of the calibration composition.

10. A method for calibrating a mass spectrometer suitable for the MALDI technique, comprising the following steps:
a) providing a calibration composition as claimed in claim 1,
b) depositing the calibration composition and a matrix suitable for the MALDI technique on at least one deposition zone of a MALDI analysis plate, in order to obtain a control zone after drying,
c) placing the MALDI analysis plate in the mass spectrometer and obtaining a mass spectrum for the control zone according to the MALDI technique, over at least one determined masses-to-charge range and using the experimental values obtained for the masses-to-charge of peaks of the spectrum corresponding to ions of so-called reference proteins of the calibration composition, as calibration values,
d) calculating a calibration relationship based on the theoretical masses-to-charge of the ions of the reference proteins and the calibration values.
